(19) United States
(12) Patent Application Publication
(10) Pub. No.: US 2024/0254513 A1
(43) Pub. Date: Aug. 1, 2024

(54) VIRAL VECTOR PRODUCTION SYSTEMS, ENGINEERED CELLS FOR VIRAL VECTOR PRODUCTION, AND METHODS OF USE THEREOF

(71) Applicant: Asimov Inc., Boston, MA (US)

(72) Inventors: Jeremy J. Gam, Boston, MA (US); Christopher S. Stach, Boston, MA (US); Alec A.K. Nielsen, Boston, MA (US)

(73) Assignee: Asimov Inc., Boston, MA (US)

(21) Appl. No.: 18/561,050

(22) PCT Filed: May 17, 2022
(86) PCT No.: PCT/US2022/029601

§ 371 Date: Nov. 15, 2023

Related U.S. Application Data
(60) Provisional application No. 63/189,771, filed on May 18, 2021.

(51) Int. Cl. C12N 15/86; C12N 9/22; C12N 15/11

(52) U.S. Cl. CPC C12N 15/86; C12N 9/22; C12N 15/11; C12N 2310/20; C12N 2740/15043; C12N 2740/15052; C12N 2750/14043; C12N 2750/14052

(57) ABSTRACT

Described herein are viral vector production systems and engineered cells for viral vector production. Also described herein are methods of using the engineered cells to produce viral vectors.

### RELATED APPLICATIONS

This application claims the benefit under 35 U.S.C. § 119 of U.S. provisional application Ser. No. 63/189,771, filed May 18, 2021, the entire contents of which are incorporated by reference herein.

### FIELD

Described herein are viral vector production systems and engineered cells for viral vector production. Also described herein are methods of using the engineered cells to produce viral vectors.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on May 17, 2022 is named A121070007WO00-SEQ-CRP and is 15,679 bytes in size.

### BACKGROUND

Viral vectors are promising therapeutics which deliver a genetic payload into target cells in order to treat a disease. A common type of payload is DNA coding for a fully functional gene of interest (GOI) to correct for a deficiency or mutation in the corresponding gene in a patient's cells. AAV vectors are produced using producer cell lines (e.g., HEK293-derived cell lines) which can produce large amounts of virus, but their growth and production rates can be affected by many factors that impact cellular health and resource allocation.

### SUMMARY

Described herein are viral vector production systems and engineered cells for viral vector production that allow one to have increased control over expression of a payload molecule. Also described herein are methods of using said systems and said engineered cells.

In some aspects, the disclosure relates to a viral vector production system comprising: (a) an engineered cell comprising a viral vector production component comprising one or more heterologous polynucleic acids that collectively encode the gene products of a viral vector; (b) a heterologous nucleic acid sequence encoding for a first expression cassette, wherein the first expression cassette comprises a nucleic acid sequence of a constitutive promoter operably linked to a nucleic acid sequence encoding a regulatory RNA; and (c) a transfer polynucleic acid comprising a central nucleic acid sequence flanked, on the 5′ and 3′ end, by a nucleic acid sequence of a viral terminal repeat.

In some embodiments, the central nucleic acid sequence of the transfer polynucleic acid comprises a nucleic acid sequence encoding a multiple cloning site. In some embodiments, the central nucleic acid sequence comprises a multiple cloning sequence flanked by a target nucleic acid sequence that complements the regulatory RNA encoded by the first expression cassette. In some embodiments, the multiple cloning sequence is flanked by a tandem repeat of a target nucleic acid sequence that complements the regulatory RNA encoded by the first expression cassette. In some embodiments, the multiple cloning sequence is flanked on the 5′ end and the 3′ end by a target nucleic acid sequence that complements the regulatory RNA encoded by the first expression cassette. In some embodiments, the central nucleic acid sequence further comprises a promoter.

In some embodiments, the central nucleic acid sequence of the transfer polynucleic acid sequence comprises a second expression cassette, wherein the second expression cassette comprises a nucleic acid sequence encoding a payload molecule operably linked to both a promoter and a target nucleic acid sequence that complements the regulatory RNA encoded by the first expression cassette.

In some embodiments, the nucleic acid sequence of the payload molecule comprises: a 5′ UTR that comprises a target nucleic acid sequence that complements the regulatory RNA encoded by the first expression cassette; a 3′ UTR that comprises a target nucleic acid sequence that complements the regulatory RNA encoded by the first expression cassette; or a combination thereof.

In some embodiments, the nucleic acid sequence of the payload molecule comprises a tandem repeat of a target nucleic acid sequence that complements the regulatory RNA encoded by the first expression cassette. In some embodiments, the first expression cassette comprises a tandem repeat of the nucleic acid sequence encoding the regulatory RNA. In some embodiments, the first expression cassette comprises a nucleic acid sequence of two or more distinct regulatory RNAs. In some embodiments, the first expression cassette further comprises a nucleic acid sequence encoding a gene product of the viral vector production component.

In some embodiments, the nucleic acid sequence encoding the gene product in the first expression cassette has: a 5′ UTR comprising the nucleic acid sequence encoding the regulatory RNA; an intron comprising the nucleic acid sequence encoding the regulatory RNA; a 3′ UTR comprising the nucleic acid sequence encoding the regulatory RNA; or a combination thereof.

In some embodiments, the first expression cassette further comprises a nucleic acid sequence encoding a selectable marker. In some embodiments, the selectable marker comprises a fluorescent protein or antibiotic resistance protein. In some embodiments, the nucleic acid sequence encoding the selectable maker in the first expression cassette has a 5′ UTR comprising the nucleic acid sequence encoding the regulatory RNA; an intron comprising the nucleic acid sequence encoding the regulatory RNA; a 3′ UTR comprising the nucleic acid sequence encoding the regulatory RNA; or a combination thereof.

In some embodiments, the viral vector production system is an AAV viral vector production system, wherein the viral vector production component comprises one or more polynucleic acids that collectively encode the gene products of an AAV vector. In some embodiments, the viral vector component comprises the nucleic acid sequences of Rep52 or Rep40; Rep78 or Rep68; E2A; E4Orf6; VARNA; VP1; VP2; VP3; and AAP. In some embodiments, the viral terminal repeats of the transfer polynucleic acid are AAV inverted tandem repeats.

In some embodiments, the viral vector production system is a lentivirus vector production system, wherein the viral vector production component comprises one or more polynucleic acids that collectively encode the gene products of a lentivirus vector. In some embodiments, the viral vector component comprises the nucleic acid sequences of VSV-G, Gag-Pol, and Rev. In some embodiments, the viral terminal repeats of the transfer polynucleic acid are lentivirus long terminal repeats.

In some embodiments, at least one of the one or more of heterologous polynucleic acids of the viral vector production component is stably integrated into the genome of the engineered cell. In some embodiments, each of the one or more of heterologous polynucleic acids of the viral vector production component is stably integrated into the genome of the engineered cell. In some embodiments, the engineered cell further comprises the heterologous nucleic acid sequence encoding for the first expression cassette.

In some embodiments, the engineered cell is derived from a HEK293 cell, a HeLa cell, a BHK cell or a Sf9 cell.

In some embodiments, the regulatory RNA of any one of the viral vector production systems described above is an shRNA or an amiRNA. In some embodiments, the nucleic acid sequence encoding the shRNA comprises a nucleic acid sequence of any one of SEQ ID NOs: 2-11. In some embodiments, the first expression cassette comprises a nucleic acid sequence encoding for a selectable marker, wherein the nucleic acid sequence encoding for the selectable marker comprises an intron having, from 5′ to 3′: (i) an intron donor site; (ii) a nucleic acid sequence encoding for the shRNA or amiRNA; and (iii) an intron acceptor site. In some embodiments, the intron comprises a tandem repeat, an shRNA cluster, or an amiRNA cluster of the nucleic acid sequence encoding for the shRNA or amiRNA.

In some embodiments, the nucleic acid sequence encoding for the selectable marker comprises: a 5′ UTR, wherein the intron of the selectable marker is located in the 5′ UTR; a 3′UTR, and wherein the intron of the selectable marker is located in the 3′ UTR; or a combination thereof. In some embodiments, the intron of the selectable marker is located in the coding region of the nucleic acid sequence encoding for the selectable marker. In some embodiments, the intron comprises the nucleic acid sequence of SEQ ID NO: 12.

In some aspects, the regulatory RNA of the viral vector production system as described above is a Cas13 guide RNA. In some embodiments, the first expression cassette comprises a constitutive promoter operably linked to a nucleic acid sequence encoding two or more Cas13 guide RNAs. In some embodiments, the viral vector production system further comprise a heterologous polynucleic acid encoding for Cas13. In some embodiments, the Cas13 is Cas13d. In some embodiments, Cas13d comprises the amino acid sequence of SEQ ID NO: 1 or an amino acid sequence having at least 80% identity to the amino acid sequence of SEQ ID NO: 1.

In some embodiments, heterologous polynucleic acid encoding for Cas13 further comprises the first expression cassette. In some embodiments, the first expression cassette comprises a nucleic acid sequence of a constitutive promoter operably linked to both the nucleic acid sequence encoding the Cas13 gRNA and the nucleic acid sequence encoding for Cas13. In some embodiments, the engineered cell further comprises the heterologous polynucleic acid encoding for Cas13.

In some aspects, an engineered cell for viral vector production comprises one or more heterologous polynucleic acids collectively comprising:
(a) a viral vector production component collectively encoding the gene products of a viral vector;
(b) a first expression cassette, wherein the first expression cassette comprises a nucleic acid sequence of a constitutive promoter operably linked to a nucleic acid sequence encoding an shRNA or amiRNA; and
(c) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the shRNA or amiRNA encoded by the first expression cassette.

In some embodiments, the nucleic acid sequence of the payload molecule comprises: a 5′ UTR that comprises a target nucleic acid sequence that complements the shRNA or amiRNA encoded by the first expression cassette; a 3′ UTR that comprises a target nucleic acid sequence that complements the shRNA or amiRNA encoded by the first expression cassette; or a combination thereof. In some embodiments, the nucleic acid sequence of the payload molecule comprises a tandem repeat of a target nucleic acid sequence that complements the shRNA or amiRNA encoded by the first expression cassette.

In some embodiments, the first expression cassette comprises a tandem repeat, shRNA cluster or amiRNA cluster of the nucleic acid sequence encoding the shRNA or amiRNA. In some embodiments, the first expression cassette comprises a nucleic acid sequence of two or more distinct shRNAs or two or more distinct amiRNAs. In some embodiments, the first expression cassette comprises a nucleic acid sequence encoding for a selectable marker, wherein the nucleic acid sequence encoding for the selectable marker comprises an intron having, from 5′ to 3′: (i) an intron donor site; (ii) a nucleic acid sequence encoding for the shRNA or amiRNA; and (iii) an intron acceptor site.

In some embodiments, the intron comprises a tandem repeat shRNA cluster or amiRNA cluster of the nucleic acid sequence encoding for the shRNA or amiRNA. In some embodiments, nucleic acid sequence encoding for the selectable marker comprises: a 5′ UTR, wherein the intron of the selectable marker is located in the 5′ UTR; a 3′UTR, wherein the intron of the selectable marker is located in the 3′ UTR; or a combination thereof.

In some embodiments, wherein the intron of the selectable marker is located in the coding region of the nucleic acid sequence encoding for the selectable marker.

In some embodiments, the intron comprises the nucleic acid sequence of SEQ ID NO: 12.

In some embodiments, the nucleic acid sequence encoding the shRNA comprises the nucleic acid sequence of any of SEQ ID NOs: 2-11.

In some embodiments, the viral vector production component comprises one or more polynucleic acids that collectively encode the gene products of an AAV vector. In some embodiments, the viral vector component comprises the nucleic acid sequences of Rep52 or Rep40; Rep78 or Rep68; E2A; E4Orf6; VARNA; VP1; VP2; VP3; and AAP. In some embodiments, the viral terminal repeats of the transfer polynucleic acid are AAV inverted tandem repeats.

In some embodiments, the viral vector production component comprises one or more polynucleic acids that collectively encode the gene products of a lentivirus vector. In some embodiments, the viral vector component comprises the nucleic acid sequences of VSV-G, Gag-Pol, and Rev. In some embodiments, the viral terminal repeats of the transfer polynucleic acid are lentivirus long terminal repeats.

In some embodiments, at least one of the one or more of heterologous polynucleic acids is stably integrated into the genome of the engineered cell. In some embodiments, each of the one or more of heterologous polynucleic acids are stably integrated into the genome of the engineered cell.

In some embodiments, the engineered cell is derived from a HEK293 cell a HeLa cell, a BHK cell or a Sf9 Cell.

In some aspects, this application discloses a method of reducing expression of a payload molecule during viral vector production in any one of the engineered cells as described above, comprising expressing the shRNA during viral vector production.

In some aspects, this application discloses an engineered cell for viral vector production comprising one or more heterologous polynucleic acids collectively comprising: (a) a viral vector production component collectively encoding the gene products of a viral vector; (b) a first expression cassette, wherein the first expression cassette comprises a nucleic acid sequence of a constitutive promoter operably linked to a nucleic acid sequence encoding a Cas13 guide RNA; (c) a nucleic acid sequence encoding Cas13; and (d) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the Cas13 guide RNA encoded by the first expression cassette.

In some embodiments, the first expression cassette comprises a constitutive promoter operably linked to a nucleic acid sequence encoding two or more Cas13 guide RNAs. In some embodiments, the Cas13 is Cas13d. In some embodiments, Cas13d comprises the amino acid sequence of SEQ ID NO: 1 or an amino acid sequence having at least 80% identity with the amino acid sequence of SEQ ID NO: 1. In some embodiments, the first expression cassette further comprises the nucleic acid sequence encoding for Cas13.

In some embodiments, the first expression cassette comprises a nucleic acid sequence of a constitutive promoter operably linked to both the nucleic acid sequence encoding the Cas13 gRNA and the nucleic acid sequence encoding for Cas13. In some embodiments, the nucleic acid sequence of the payload molecule comprises: a 5′ UTR that comprises a target nucleic acid sequence that complements the Cas13 guide RNA encoded by the first expression cassette; a 3′ UTR that comprises a target nucleic acid sequence that complements the Cas13 guide RNA encoded by the first expression cassette; or a combination thereof.

In some embodiments, the nucleic acid sequence of the payload molecule comprises a tandem repeat of a target nucleic acid sequence that complements the Cas13 guide RNA encoded by the first expression cassette. In some embodiments, the first expression cassette comprises a tandem repeat of the nucleic acid sequence encoding the Cas13 guide RNA.

In some embodiments, the first expression cassette comprises a nucleic acid sequence of two or more distinct Cas13 guide RNAs.

In some embodiments, the viral vector production component comprises one or more polynucleic acids that collectively encode the gene products of an AAV vector. In some embodiments, the viral vector component comprises the nucleic acid sequences of Rep52 or Rep40; Rep78 or Rep68; E2A; E4Orf6; VARNA; VP1; VP2; VP3; and AAP. In some embodiments, the viral terminal repeats of the transfer polynucleic acid are AAV inverted tandem repeats.

In some embodiments, the viral vector production component comprises one or more polynucleic acids that collectively encode the gene products of a lentivirus vector. In some embodiments, the viral vector component comprises the nucleic acid sequences of VSV-G, Gag-Pol, and Rev. In some embodiments, the viral terminal repeats of the transfer polynucleic acid are lentivirus long terminal repeats.

In some embodiments, at least one of the one or more of heterologous polynucleic acids is stably integrated into the genome of the engineered cell. In some embodiments, each of the one or more of heterologous polynucleic acids are stably integrated into the genome of the engineered cell.

In some embodiments, the engineered cell is derived from a HEK293 cell, a HeLa cell, a BHK cell or a Sf9 Cell.

In some aspects, this disclosure related to a method of reducing expression of a payload molecule during viral vector production in any one of the engineered cells described herein, comprising expressing the Cas13 and the Cas13 guide RNA during viral vector production.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows an exemplary schematic of shRNA-mediated knockdown of an AAV payload molecule (or gene of interest, “GOI”). 1) RNAs are transcribed corresponding to the GOI and also a gene (here Neo-TagBFP) with an shRNA in the 3′ UTR. The shRNA (loop) is flanked by intron donor and acceptor sites (asterisks). The GOI is flanked by tandem repeats of shRNA target sites (shaded boxes) in the 5′ and 3′ UTRs. 2) The shRNA sequence is spliced, freeing the shRNA hairpin. 3) The shRNA hairpin is processed by RNAi machinery and the guide strand is incorporated into an RNA-induced silencing complex (RISC). 4) RISC with the guide strand binds to the GOI mRNA 5) RISC can cleave the GOI mRNA and/or repress its translation. Reduction in GOI mRNA available for translation results in decreased levels of GOI protein.

FIGS. 2A-2B show a comparison of payload molecule (EGFP) knockdown in cells having or lacking an shRNA expression plasmid. FIG. 2A shows fluorescence images of AAV producer cells prior to harvesting AAV and AAV infectious titers, with and without addition of EGFP shRNAs. FIG. 2B shows quantification of AAV titers.

FIG. 3 shows a comparison of Immunoglobulin (IG)-EYFP payload molecules containing FF5 target sites in either the 5′ UTR or 3′ UTR, IG-EYFP payload molecule lacking FF5 target sites, and a EGFP payload molecule lacking FF5 target sites. On the left are fluorescence geometric means of EYFP or EGFP measured within the transfected cells being used to produce AAV. On the right are virus titers measured using a transduction assay. Transfection with FF5 shRNA (blue bars) results in a marked reduction of the payload molecule in the AAV-producing cells for constructs bearing FF5 target sites, but still produces similar or better virus titers compared to the controls where FF5 shRNA is not transfected (red bars).

FIG. 4 shows an exemplary schematic of Cas13-mediated knockdown of an AAV payload molecule (or gene of interest, “GOI”). The knockdown system consists of an array of target sequences placed in the 5′UTR, 3′UTR or both regions recognized by the Cas13d-crRNA complex.

### DETAILED DESCRIPTION

The inventors of the instant disclosure have appreciated that expression of a payload molecule (i.e., gene of interest or “GOI”) of a viral vector during viral vector production can divert resources away from producing viral vectors and towards producing RNA and protein of the payload. The inventors have also appreciated that some payload molecules may be toxic, further impacting the growth and productivity of the producer cells. Described herein are viral vector production systems and engineered cells for viral vector production that allow one to have increased control over expression of a payload molecule during viral vector production. Also described herein are methods of using said engineered cells.

### I. Viral Vector Production Systems

In some aspects, the disclosure relates to viral vector production systems. A viral vector production system, as described herein, comprises one or more polynucleic acids collectively comprising: (a) a viral vector production component; (b) a first expression cassette comprising a nucleic acid sequence of a promoter operably linked to a nucleic acid sequence encoding a regulatory RNA; and (c) a transfer polynucleic acid.

As used herein, the term “viral vector production component” refers to one or more polynucleic acids that collectively encode the gene products required for generation of viral vectors in a recombinant host cell. Several types of viral vectors (including components required for their production) have been described previously, including adenovirus vectors, adeno-associated virus (AAV) vectors, lentivirus vectors, retrovirus vectors, and herpes-simplex virus vectors. The viral vector production systems described herein may comprise a viral vector production component encoding gene products required for the production of any of these previously described viral vectors.

In some embodiments, a viral vector production component comprises one or more polynucleotides that collectively encode the gene products required to generate an AAV vector in a recombinant host cell. Exemplary AAV gene products include Rep52, Rep40, Rep78, Rep68, E2A, E4Orf6, VARNA, VP1, VP2, VP3, AAP and MAAP or a functional variant thereof. The Rep gene products (comprising Rep52, Rep40, Rep78 and Rep68) are involved in AAV genome replication. The E2A gene product is involved in aiding DNA synthesis processivity during AAV replication. The E4Orf6 gene product supports AAV replication. The VARNA gene product plays a role in regulating translation. The CAP gene products (comprising VP1, VP2, VP3) encode viral capsid proteins. The AAP gene product plays a role in capsid assembly. MAAP is a frameshifted VP1 protein and appears to play a role in the viral capsid as described in Ogden et al. Science 366.6469 (2019): 1139-1143, which is incorporated by reference in its entirety.

As used herein, the term “functional variant” refers a gene product that comprises a modified nucleic acid or amino acid sequence compared to a wildtype sequence and is capable of performing the function (e.g. enzymatic, regulation, or binding) of the wildtype type gene product. For example, a functional variant of Rep52 is still capable of functioning in AAV genome replication.

In some embodiments, a viral vector component comprises one or more polynucleotides that collectively encode the gene products: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP 1 (or a functional variant thereof); VP2 (or a functional variant thereof); VP3 (or a functional variant thereof); and AAP (or a functional variant thereof). In some embodiments, a viral vector component comprises one or more polynucleotides that collectively encode the gene products: Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof (e.g. SEQ ID NO: 23), VARNA (or a functional variant thereof), VP1 (or a functional variant thereof), VP2 (or a functional variant thereof), VP3 (or a functional variant thereof), and AAP (or a functional variant thereof).

Exemplary Functional Variant of E4Orf6 with Splice Site Removed:

(SEQ ID NO: 23)
atgactacgtccggcgttccatttggcatgacactacgaccaacacgat
ctcggttgtctcggcgcactccgtacagtagggatcgcctacctccttt
tgagacagagacccgcgctaccatactggaggatcatccgctgctgccc
gaatgtaacactttgacaatgcacaaTgtTTCCtacgtgcgaggtcttc
cctgcagtgtgggatttacgctgattcaggaatgggttgttccctggga
tatggttctgacgcgggaggagcttgtaatcctgaggaagtgtatgcac
gtgtgcctgtgttgtgccaacattgatatcatgacgagcatgatgatcc
atggttacgagtcctgggctctccactgtcattgttccagtcccggttc
cctgcagtgcatagccggcgggcaggttttggccagctggtttaggatg
gtggtggatggcgccatgtttaatcagaggtttatatggtaccgggagg
tggtgaattacaacatgccaaaagaggtaatgtttatgtccagcgtgtt
tatgaggggtcgccacttaatctacctgcgcttgtggtatgatggccac
gtgggttctgtggtccccgccatgagctttggatacagcgccttgcact
gtgggattttgaacaatattgtggtgctgtgctgcagttactgtgctga
tttaagtgagatcagggtgcgctgctgtgcccggaggacaaggcgtctc
atgctgcgggcggtgcgaatcatcgctgaggagaccactgccatgttgt
attcctgcaggacggagcggcggcggcagcagtttattcgcgcgctgct
gcagcaccaccgccctatcctgatgcacgattatgactctacccccatg
TAGtaa

In some embodiments, a viral vector production component comprises one or more polynucleotides that collectively encode the gene products required to generate a lentivirus vector in a recombinant host cell. Exemplary lentivirus gene products include: VSV-G, Gag-Pol, and Rev. In some embodiments, a viral vector comprises one or more polynucleotides that collectively encode the gene products: VSV-G (or a functional variant thereof), Gag-Pol (or a functional variant thereof), and Rev (or a functional variant thereof).

In some embodiments, the viral vector component is (i.e., the gene products of the viral vector component are) encoded on a single polynucleic acid. In other embodiments, multiple polynucleic acids collectively comprise the viral vector component (i.e., at least two of the gene products of the viral vector component are encoded on different polynucleic acids). For example, a viral vector component may comprise at least 2, at least 3, at least 4, or at least 5 polynucleic acids. In some embodiments, a viral vector component comprises 2, 3, 4, or 5 polynucleic acids.

In addition to the viral vector component, the viral vector production systems described herein comprise a first expression cassette comprising a nucleic acid sequence of a promoter operably linked to a nucleic acid sequence encoding a regulatory RNA.

As expounded upon below (Parts IA and IB), exemplary regulatory RNAs include shRNAs and Cas13 guide RNAs.

The first expression cassette may comprise a tandem repeat of the nucleic acid sequence encoding the regulatory RNA. For example, in some embodiments, a tandem repeat may comprise at least 2, at least 3, at least 4, at least 5, at least 6, at least 7, at least 8, at least 9, or at least 10 copies of the nucleic acid sequence encoding the regulatory RNA. In some embodiments, a tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of the nucleic acid sequence encoding the regulatory RNA. In some embodiments, a tandem repeat comprises 2, 3, 4, 5, 6, 7, 8, 9, or 10 copies of the nucleic acid sequence encoding the regulatory RNA.

The first expression cassette may comprise two or more tandem repeats of the nucleic acid sequence encoding the regulatory RNA. For example, in some embodiments, the first expression cassette comprises at least 2, at least 3, at least 4, or at least 5 tandem repeats of the nucleic acid sequence encoding the regulatory RNA. In some embodiments, the first expression cassette comprises 2-3, 2-4, 2-5, 3-4, 3-5, or 4-5 tandem repeats of the nucleic acid sequence encoding the regulatory RNA. In some embodiments, the first expression cassette comprises 2, 3, 4, or 5 tandem repeats of nucleic acid sequence encoding the regulatory RNA.

The first expression cassette may comprise a cluster of the nucleic acid sequence encoding the regulatory RNA (e.g., an shRNA cluster or an artificial miRNA cluster). As used herein, the term cluster refers to a polynucleic acid encoding a set of two or more miRNAs that are physically adjacent (i.e. within about 10 kilobases), transcribed in the same orientation, and are not separated by a transcriptional unit or an miRNA in the opposite orientation as described in Lai, X., and J. Vera. “MicroRNA clusters.” Encyclopedia of Systems Biology. New York: Springer (2013), which is incorporated by reference in its entirety. Exemplary miRNA clusters include but are not limited to, miR-30e, miR-30c-1, miR-214, miR-199a-2, miR-215, miR-194-1, miR-217, miR-216, miR-15b, miR-16-2, miR-143, miR-145, miR-25, miR-93, miR-106b, miR-23b, miR-27b, miR-24-1, miR-181a, miR-181b-2, miR-34b; miR-34c, miR-125b-1, let-7a-2, miR-100, miR-16-1, miR-15a, miR-17, miR-18, miR-19a, miR-20, miR-19b-1, miR-92-1, miR-299, miR-323, miR-329, miR-134, miR-154, miR-133a-1, miR-1-2, miR-99b, let-7e, miR-125a, miR-133a-2, miR-1-1, miR-99a, let-7c, miR-125b-2, miR-98, let-7f-2, miR-105-1, and miR-105-2. In some embodiments, the cluster encodes 2, 3, 4, 5, 6, 7, 8, 9, 10, or more than 10 miRNAs. An shRNA cluster or an artificial miRNA cluster, as described herein, refers to a cluster where the hairpins of the miRNAs have been replaced with hairpins of the shRNAs or artificial miRNAs as described herein (e.g. an shRNA hairpin that targets a payload gene). Methods of producing amiRNA clusters are well known in the art and are described in Bhaskaran, Vivek, et al. Nature protocols 14.12 (2019): 3538-3553, which is incorporated by reference in its entirety. In some embodiments, the shRNA or amiRNA cluster comprises an shRNA or amiRNA hairpin that is not naturally occurring. In some embodiments, the 5′-most and 3′-most flanking areas of the shRNA cluster or amiRNA cluster are replaced with flanking areas of a different miRNA cluster producing a chimeric shRNA cluster or chimeric amiRNA cluster.

The first expression cassette may comprise nucleic acid sequences of distinct regulatory RNAs (i.e., regulatory RNAs having distinct nucleic acid sequences). For example, in some embodiments, the first expression cassette comprises nucleic acid sequences of at least 2, at least 3, at least 4, at least 5, at least 6, at least 7, at least 8, at least 9, or at least 10 distinct regulatory RNAs. In some embodiments, the first expression cassette comprises nucleic acid sequences of 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct regulatory RNAs. In some embodiments, the first expression cassette comprises nucleic acid sequences of 2, 3, 4, 5, 6, 7, 8, 9, or 10 distinct regulatory RNAs.

As described herein, a promoter is “operably linked” to a nucleic acid coding sequence when the position of the promoter relative to the nucleic acid coding sequence is such that binding of a transcriptional activator to the promoter can induce expression of the coding sequence.

A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110(28): 11284-89; Pub. No.: US 2014/377861 A1; Qin, Jane Yuxia, et al. “PloS one 5.5 (2010): e10611—the entireties of which are incorporated herein by reference.

Alternatively, a promoter may be an inducible promoter (i.e., only activates transcription under specific circumstances). An inducible promoter may be, for example, a chemically inducible promoter, a temperature inducible promoter, or a light inducible promoter. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3(12): 880-91; Liang et al., Sci. Signal. 2011 Mar. 15; 4(164): rs2; U.S. Pat. Nos. 7,745,592 B2; 7,935,788 B2—the entireties of which are incorporated herein by reference.

In some embodiments, the promoter of the first expression cassette is a constitutive promoter, such as a CMV promoter, an EF1a promoter, an SV40 promoter, a UBC promoter, a U6 promoter, or a PGK promoter.

In some embodiments, the promoter of the first expression cassette is an inducible promoter, such as a chemically inducible promoter, a temperature inducible promoter, or a light inducible promoter. In some embodiment, the inducible promoter is a tetracycline/doxycycline inducible promoter, a cumate inducible promoter, an ABA inducible promoter, a CRY2-CIB1 inducible promoter, a DAPG inducible promoter, or a mifepristone inducible promoter.

In some embodiments, the first expression cassette further comprises a nucleic acid sequence encoding a gene product of the viral vector production component described above. For example, in embodiments wherein the viral vector production component is an AAV viral vector component, the first expression cassette may further comprise a nucleic acid sequence encoding Rep52, Rep40, Rep78, Rep68, E2A, E4Orf6, VARNA, VP1, VP2, VP3, AAP, or a combination thereof. Similarly, in embodiments wherein the viral vector production component is a lentiviral vector component, the first expression cassette may further comprise a nucleic acid sequence encoding for VSV-G, Gag-Pol, Rev, or a combination thereof.

In some embodiments, the promoter of the first expression cassette is operably linked to both the nucleic acid sequence encoding the regulatory RNA and the nucleic acid sequence encoding a gene product of the viral vector production component. For example, in some embodiments, the nucleic acid sequence encoding the gene product has a 5′ UTR, an intron, and/or a 3′ UTR comprising the nucleic acid sequence encoding a regulatory RNA.

In some embodiments, the first expression cassette further comprises a nucleic acid sequence encoding a selectable marker. As used herein, the term “selectable marker” refers to a protein that—when introduced into or expressed in a cell—confers a trait that is suitable for selection.

A selectable marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selectable marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

In some embodiments, the promoter of the first expression cassette is operably linked to both the nucleic acid sequence encoding the regulatory RNA and the nucleic acid sequence encoding a selectable marker. For example, in some embodiments, the nucleic acid sequence encoding the selectable maker has a 5′ UTR, an intron, and/or a 3′ UTR comprising the nucleic acid sequence encoding a regulatory RNA.

In addition to the viral vector component and the first expression cassette, the viral vector production systems described herein comprise a transfer polynucleic acid. The transfer polynucleic acids described herein comprise a central nucleic acid sequence flanked, on the 5′ end and the 3′ end, by a nucleic acid sequence of a viral terminal repeat. As used herein the term “viral terminal repeat” refers to a nucleic acid sequence required for polynucleic acid integration of a viral vector payload into a host cell genome. Exemplary viral terminal repeats are known to those having ordinary skill in the art.

For example, in embodiments wherein the viral vector production component is an AAV viral vector component, a transfer polynucleic acid may comprise a central nucleic acid sequence flanked, on the 5′ end and the 3′ end, by a nucleic acid sequence of an AAV inverted tandem repeat (“ITR”). Exemplary AAV ITRs are known to those having ordinary skill in the art.

In embodiments wherein the viral vector production component is a lentiviral vector component, a transfer polynucleic acid may comprise a central nucleic acid sequence flanked, on the 5′ end and the 3′ end, by a nucleic acid sequence of a lentivirus long tandem repeat (LTR). Exemplary lentivirus LRTs are known to those having ordinary skill in the art.

The central nucleic acid of a transfer polynucleic acid may comprise a target nucleic acid sequence that complements the regulatory RNA encoded by the first expression cassette. As described herein, a target nucleic acid sequence “complements” a regulatory RNA, when it is capable of being bound by the regulatory RNA (i.e., capable of hybridizing with the regulatory RNA) under physiological conditions of a host cell. A target nucleic acid sequence is said to have 100% complementarity to a regulatory RNA when it comprises a nucleic acid sequence that is a reverse compliment of a regulatory RNA. In some embodiments, a target nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementarity to a regulatory RNA. In some embodiment a target nucleic acid sequence has 85-100%, 90-100%, 95-100%, 96-100%, 97-100%, 98-100%, or 99-100% complementarity to a regulatory RNA.

Alternatively, or in addition, a central nucleic acid of a transfer polynucleic acid may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule—into the transfer polynucleic acid prior to the generation of viral vectors in a host cell. In some embodiments, the nucleic acid sequence of the multiple cloning site is flanked (on the 5′ end, the 3′ end, or both the 5′ end and the 3′ end) by a target nucleic acid sequence that complements the regulatory RNA encoded by the first expression cassette—all being comprised within the central nucleic acid. For example, a central nucleic acid may comprise a 5′UTR sequence and/or a 3′UTR sequence comprising a target nucleic acid sequence (that complements the regulatory RNA encoded by the first expression cassette) which can be operably linked to a gene of interest that is cloned into the multiple cloning site. In some embodiments, a central nucleic acid further comprises the nucleic acid sequence of a promoter (constitutive or inducible, as described herein). For example, in some embodiments, a transfer polynucleic acid comprises, from 5′ to 3′: (i) a nucleic acid sequence of a viral terminal repeat; (ii) a nucleic acid sequence of a promoter; (iii) a nucleic acid sequence of a multiple cloning site that is flanked (on the 3′ end, the 5′ end, or both the 5′ end and the 3′ end) by a target nucleic acid sequence that complements the regulatory RNA encoded by the first expression cassette; and (iv) a nucleic acid sequence of a viral terminal repeat.

Alternatively, or in addition, a central nucleic acid of a transfer polynucleic acid may comprise an expression cassette comprising a promoter (constitutive or inducible, as described herein) and a target nucleic acid sequence that complements a regulatory RNA encoded by the first expression cassette, both of which are operably linked to a nucleic acid sequence encoding a payload molecule. In some embodiments, the nucleic acid sequence encoding the payload molecule comprises: a 5′UTR that comprises a target nucleic acid sequence; a coding sequence comprising a target nucleic acid sequence; a 3′UTR that comprises a target nucleic acid sequence; or a combination thereof.

A central nucleic acid may comprise a tandem repeat of a target nucleic acid sequence (i.e., a nucleic acid sequence that complements a regulatory RNA encoded by the first expression cassette). In some embodiments, a tandem repeat may comprise at least 2, at least 3, at least 4, at least 5, at least 6, at least 7, at least 8, at least 9, or at least 10 copies of a target nucleic acid sequence. In some embodiments, a tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of a target nucleic acid sequence. In some embodiments, a tandem repeat comprises 2, 3, 4, 5, 6, 7, 8, 9, 10 copies of a target nucleic acid sequence.

A central nucleic acid may comprise two or more tandem repeats of a target nucleic acid sequence (i.e., a nucleic acid sequence that complement a regulatory RNA encoded by the first expression cassette). In some embodiments, a central nucleic acid comprises at least 2, at least 3, at least 4, or at least 5 tandem repeats of the nucleic acid sequence encoding the regulatory RNA. In some embodiments, a central nucleic acid comprises 2-3, 2-4, 2-5, 3-4, 3-5, or 4-5 tandem repeats of the nucleic acid sequence encoding the regulatory RNA. In some embodiments, a central nucleic acid comprises 2, 3, 4, or 5 tandem repeats of nucleic acid sequence encoding the regulatory RNA.

In embodiments wherein the first expression cassette comprises distinct regulatory RNAs (i.e., regulatory RNAs having distinct nucleic acid sequences), a central nucleic acid may comprise distinct target nucleic acid sequences. For example, in some embodiments, a central nucleic acid comprises nucleic acid sequences of at least 2, at least 3, at least 4, at least 5, at least 6, at least 7, at least 8, at least 9, or at least 10 distinct target nucleic acid sequences. In some embodiments, a central nucleic acid comprises nucleic acid sequences of 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct target nucleic acid sequences. In some embodiments, a central nucleic acid comprises nucleic acid sequences of 2, 3, 4, 5, 6, 7, 8, 9, or 10 distinct target nucleic acid sequences.

In some embodiments, a viral vector production system described herein comprises an engineered cell. The engineered cell may comprise any part (and any combination of parts) of the viral vector production systems described herein.

For example, an engineered cell may comprise at least a portion of the viral vector production component. For example, and as described above, a viral vector production component may comprise multiple polynucleic acids. In such embodiments, an engineered cell comprises one or more of said multiple polynucleic acids—each of which may be located extra-chromosomally or stably integrated into the genome of the engineered cell. In some embodiments, an engineered cell comprises the entire viral vector production component.

Alternatively, or in addition, an engineered cell may comprise the first expression cassette of the viral production system.

Alternatively, or in addition, an engineered cell may comprise the transfer polynucleic acid of the viral production system.

In some embodiments, a viral vector production system comprises: (a) an engineered cell comprising a viral vector production component comprising one or more heterologous polynucleic acids that collectively encode the gene products of a viral vector; (b) a heterologous nucleic acid sequence encoding for a first expression cassette, wherein the first expression cassette comprises a nucleic acid sequence of a constitutive promoter operably linked to a nucleic acid sequence encoding a regulatory RNA; and (c) a transfer polynucleic acid comprising a central nucleic acid sequence flanked, on the 5′ and 3′ end, by a nucleic acid sequence of a viral terminal repeat.

A. Production Systems Having an shRNA Regulatory RNA.

In some embodiments, the regulatory RNA of a viral vector production system is an shRNA. Small hairpin RNAs (“shRNAs”) are sequences that mimic microRNAs, which downregulate RNA transcripts with sufficient complementarity to the microRNA sequence. shRNAs are transcribed by Pol II or Pol III promoters and processed and integrated into an RNA-induced silencing complex (RISC). In some embodiments, the regulatory RNA of a viral vector production system is an amiRNA (artificial microRNA). Artificial miRNA are naturally occurring pri-miRNA sequences that have been modified to comprise sequences that direct downregulation of a target gene (e.g. a payload gene). shRNAs and amiRNAs can be designed to be complementary to the payload molecule (GOI) coding sequence and/or UTR (5′ UTR or 3′ UTR). Specific target sequences can be incorporated into, for example, the UTR sequences of a payload molecule.

The productions systems having an shRNA as a regulatory RNA may have any of the embodiments described above (Part I). The productions systems having an amiRNA as a regulatory RNA may have any of the embodiments described above (Part I).

In some embodiments, the nucleic acid sequence encoding for the shRNA comprises the nucleic acid sequence of any one of SEQ ID NOs: 2-11. In some embodiments, the shRNA or amiRNA targets the sequence of any one of SEQ ID NO: 13-22.

In some embodiments, the first expression cassette comprises a nucleic acid sequence encoding for a selectable marker (as described herein), wherein the nucleic acid sequence encoding for the selectable marker comprises an intron having, from 5′ to 3′: (i) an intron donor site; (ii) a nucleic acid sequence encoding for the shRNA; and (iii) an intron acceptor site.

In some embodiments, the shRNA is operably linked to a PolIII promoter (e.g. a U6 promoter). In some embodiments, the amiRNA is operably linked to a PolIII promoter (e.g. a U6 promoter).

In some embodiments, the intron comprises a tandem repeat of the nucleic acid sequence encoding for the shRNA. In some embodiments, the tandem repeat comprises 2, 3, 4, 5, 6, 7, 8, 9, or 10 copies of the nucleic acid sequence encoding for the shRNA. In some embodiments, the tandem repeat comprises at least 2, at least 3, at least 4, or at least 5 copies of the nucleic acid sequence encoding for the shRNA. In some embodiments, the tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of the nucleic acid sequence encoding for the shRNA.

In some embodiments, the intron comprises an shRNA cluster of the nucleic acid sequence encoding for the shRNA. In some embodiments, the shRNA cluster comprises 2, 3, 4, 5, 6, 7, 8, 9, or 10 copies of the nucleic acid sequence encoding for the shRNA. In some embodiments, the shRNA cluster comprises at least 2, at least 3, at least 4, or at least 5 copies of the nucleic acid sequence encoding for the shRNA. In some embodiments, the shRNA cluster comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of the nucleic acid sequence encoding for the shRNA.

In some embodiments, the intron comprises an amiRNA cluster of the nucleic acid sequence encoding for the amiRNA. In some embodiments, the amiRNA cluster comprises 2, 3, 4, 5, 6, 7, 8, 9, or 10 copies of the nucleic acid sequence encoding for the amiRNA. In some embodiments, the amiRNA cluster comprises at least 2, at least 3, at least 4, or at least 5 copies of the nucleic acid sequence encoding for the amiRNA. In some embodiments, the amiRNA cluster comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of the nucleic acid sequence encoding for the amiRNA.

In some embodiments, the nucleic acid sequence encoding for the selectable marker comprises: a 5′ UTR, wherein the intron of the selectable marker is located in the 5′ UTR; a 3′ UTR, wherein the intron of the selectable marker is located in the 3′ UTR; or a combination thereof. In some embodiments, the intron of the selectable marker is located in the coding region of the nucleic acid sequence encoding for the selectable marker.

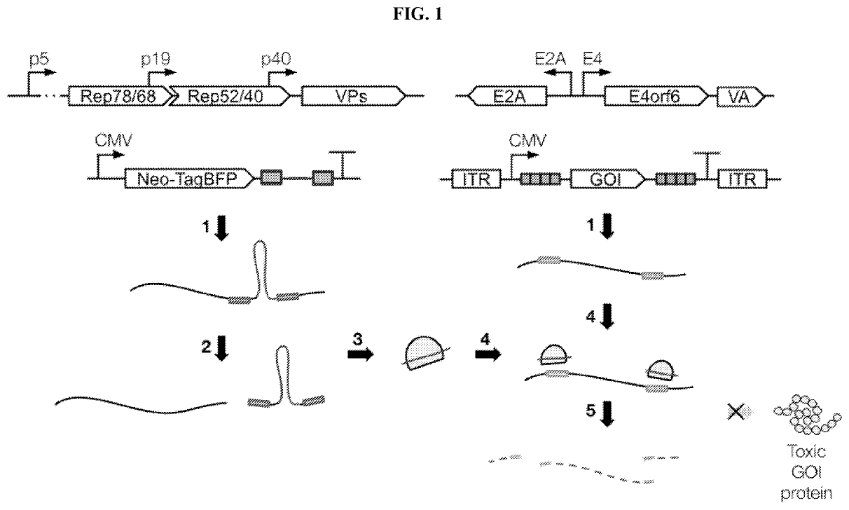

In some embodiments, the viral vector production system is as depicted in FIG. 1.

In some embodiments, the intron comprises the nucleic acid sequence of AGgtaagtNNNNTACTTTAGGACCCTTTTTTTTCCacagGT (SEQ ID NO: 12), where the “NNNN” comprises the targeting sequence of the shRNA. In some embodiments, “NNNN” comprises any one of SEQ ID NOs: 2-11.

### B. Production Systems Having a Cas13 Guide RNA Regulatory RNA.

In some embodiments, the regulatory RNA of a viral vector production system is a Cas13 guide RNA. Cas13 is a programmable RNA-guided, RNA-targeting Cas protein with nuclease activity that allows for targeted mRNA knockdown without altering the coding DNA sequence of a gene. Cas13 is guided to target RNAs by a guide RNA that complements the target sequence. Target recognition leads RNA-RNA hybridization and cleavage of the target RNA. Guide RNAs can be designed to be complementary to the payload molecule (GOI) coding sequence and/or UTR (5′ UTR or 3′ UTR). Specific target sequences can be incorporated into, for example, the UTR sequences of a payload molecule. Cas13 can be employed to knockdown payload molecule (“GOI”) protein levels through RNA cleavage without modifying the coding DNA sequence. Cas13 does not exhibit a protospacer flanking sequence requirement allowing for targeting of any sequence within the transcribed region.

The production systems having a Cas13 guide RNA as a regulatory RNA may have any of the embodiments described above (Part I).

In some embodiments, the first expression cassette comprises a constitutive promoter operably linked to a nucleic acid sequence encoding two or more distinct Cas13 guide RNAs. In some embodiments, the two or more distinct gRNAs are comprised in a guide RNA array selected from the group consisting of the native guide RNA array, a Ribozyme self-cleavage guide RNA array, at Cys4 guide RNA array, or a tRNA guide RNA array as described in McCarty, Nicholas S., et al. “Nature communications 11.1 (2020): 1-13, which is incorporated by reference in its entirety. In some embodiments, the first expression cassette comprises a nucleic acid sequence encoding for 2, 3, 4, 5, 6, 7, 8, 9, or 10 distinct Cas13 guide RNAs. In some embodiments, the first expression cassette comprises a nucleic acid sequence encoding for at least 2, at least 3, at least 4, or at least 5 distinct Cas13 guide RNAs. In some embodiments, the first expression cassette comprises a nucleic acid sequence encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct Cas13 guide RNAs.

In some embodiments, a viral vector production system further comprises a heterologous polynucleic acid encoding for Cas13. Exemplary Cas13 proteins are known to those having ordinary skill in the art and include, but are not limited to, Cas13a, Cas13b, Cas13c, and Cas13d. In some embodiments, a viral vector production system comprises a heterologous polynucleic acid encoding for Cas13a. In some embodiments, a viral vector production system comprises a heterologous polynucleic acid encoding for Cas13b. In some embodiments, a viral vector production system comprises a heterologous polynucleic acid encoding for Cas13c. In some embodiments, a viral vector production system comprises a heterologous polynucleic acid encoding for Cas13d.

In some embodiments, the Cas13 comprises the amino acid sequence of SEQ ID NO: 1 or an amino acid sequence of a functional variant having at least 80%, at least 85%, at least 90%, at least 95%, at least 96%, at least 97%, at least 98%, or at least 99% amino acid identity with the amino acid sequence of SEQ ID NO: 1. As used herein, the term “functional variant”—in the context of a Cas13 protein—refers to a variant having at least 50% endonuclease activity relative to the wild type Cas13 protein.

Methods of determining the extent of identity between two sequences (e.g., two amino acid sequences or two polynucleic acids) are known to those having ordinary skill in the art. One exemplary method is the use of Basic Local Alignment Search Tool (BLAST®) software with default parameters (blast.ncbi.nlm.nih.gov/Blast.cgi).

In some embodiments, the heterologous polynucleic acid encoding for Cas13 further comprises the first expression cassette. In some embodiments, the first expression cassette comprises a nucleic acid sequence of a constitutive promoter operably linked to both the nucleic acid sequence encoding the Cas13 gRNA and the nucleic acid sequence encoding for Cas13.

In some embodiments, an engineered cell comprises the heterologous polynucleic acid encoding for Cas13.

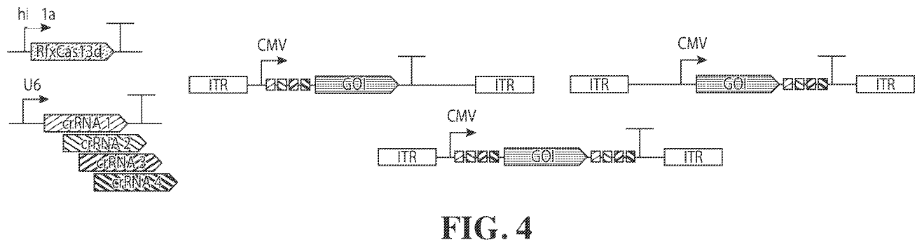

In some embodiments, the viral vector production system is as depicted in FIG. 4.

### II. Engineered Cells for Viral Vector Production

In some aspects, the disclosure relates to engineered cells for viral vector production. These engineered cells may comprise any combination of parts of the viral vector production systems described above. Exemplary engineered cells for viral vector production are provided below.

A. Engineered Cells Comprising an shRNA Regulatory RNA.

In some embodiments, an engineered cell for viral vector production comprises one or more heterologous polynucleic acids collectively comprising: (a) a viral vector production component collectively encoding the gene products of a viral vector; (b) a first expression cassette, wherein the first expression cassette comprises a nucleic acid sequence of a constitutive promoter operably linked to a nucleic acid sequence encoding an shRNA; and (c) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the shRNA encoded by the first expression cassette.

In some embodiments, the nucleic acid sequence of the payload molecule comprises a 5′ UTR that comprises a target nucleic acid sequence that complements the shRNA encoded by the first expression cassette; a 3′ UTR that comprises a target nucleic acid sequence that complements the shRNA encoded by the first expression cassette; or a combination thereof.

In some embodiments, the nucleic acid sequence of the payload molecule comprises a tandem repeat of a target nucleic acid sequence that complements the shRNA encoded by the first expression cassette. In some embodiments, a tandem repeat may comprise at least 2, at least 3, at least 4, at least 5, at least 6, at least 7, at least 8, at least 9, or at least 10 copies of a target nucleic acid sequence. In some embodiments, a tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of a target nucleic acid sequence. In some embodiments, a tandem repeat comprises 2, 3, 4, 5, 6, 7, 8, 9, 10 copies of a target nucleic acid sequence.

In some embodiments, the first expression cassette comprises a tandem repeat of the nucleic acid sequence encoding the shRNA. In some embodiments, the tandem repeat comprises 2, 3, 4, 5, 6, 7, 8, 9, or 10 copies of the nucleic acid sequence encoding for the shRNA. In some embodiments, the tandem repeat comprises at least 2, at least 3, at least 4, or at least 5 copies of the nucleic acid sequence encoding for the shRNA. In some embodiments, the tandem repeat comprise 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of the nucleic acid sequence encoding for the shRNA.

In some embodiments, the nucleic acid sequence encoding the shRNA comprises the nucleic acid sequence of any of SEQ ID NOs: 2-11.

In some embodiments, the first expression cassette comprises a nucleic acid sequence of two or more distinct shRNAs. For example, in some embodiments, the first expression cassette comprises a nucleic acid sequence encoding for 2, 3, 4, 5, 6, 7, 8, 9, or 10 distinct shRNAs. In some embodiments, the first expression cassette comprises a nucleic acid sequence encoding for at least 2, at least 3, at least 4, or at least 5 distinct shRNAs. In some embodiments, the first expression cassette comprises a nucleic acid sequence encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct shRNAs.

In some embodiments, the first expression cassette comprises a nucleic acid sequence encoding for a selectable marker, wherein the nucleic acid sequence encoding for the selectable marker comprises an intron having, from 5′ to 3′: (i) an intron donor site; (ii) a nucleic acid sequence encoding for the shRNA; and (iii) an intron acceptor site.

In some embodiments, the intron comprises a tandem repeat of the nucleic acid sequence encoding for the shRNA.

In some embodiments, the nucleic acid sequence encoding for the selectable marker comprises: a 5′ UTR, wherein the intron of the selectable marker is located in the 5′ UTR; a 3′ UTR, wherein the intron of the selectable marker is located in the 5′ UTR; or a combination thereof. In some embodiments, the intron of the selectable marker is located in the coding region of the nucleic acid sequence encoding for the selectable marker.

In some embodiments, the intron comprises the nucleic acid sequence of SEQ ID NO: 12.

In some embodiments, the viral vector production component comprises one or more polynucleic acids that collectively encode the gene products of an AAV vector. In some embodiments, the viral vector component comprises the nucleic acid sequences of Rep52 or Rep40; Rep78 or Rep68; E2A; E4Orf6; VARNA; VP1; VP2; VP3; and AAP. In some embodiments, the viral terminal repeats of the transfer polynucleic acid are AAV inverted tandem repeats.

In some embodiments, the viral vector production component comprises one or more polynucleic acids that collectively encode the gene products of a lentivirus vector. In some embodiments, the viral vector component comprises the nucleic acid sequences of VSV-G, Gag-Pol, and Rev. In some embodiments, the viral terminal repeats of the transfer polynucleic acid are lentivirus long terminal repeats.

In some embodiments, the engineered cell comprises a viral vector production system as depicted in FIG. 1.

In some embodiments, at least one of the one or more of heterologous polynucleic acids is stably integrated into the genome of the engineered cell. In some embodiments, each of the one or more of heterologous polynucleic acids are stably integrated into the genome of the engineered cell.

In some embodiments, the engineered cell is derived from a HEK293 cell or a HeLa cell, a BHK cell, or a Sf9 cell.

### B. Engineered Cells Comprising a Cas13 Guide RNA Regulatory RNA.

In some embodiments, an engineered cell for viral vector production comprises one or more heterologous polynucleic acid collectively comprising: (a) a viral vector production component collectively encoding the gene products of a viral vector; (b) a first expression cassette, wherein the first expression cassette comprises a nucleic acid sequence of a constitutive promoter operably linked to a nucleic acid sequence encoding a Cas13 guide RNA; (c) a nucleic acid sequence encoding Cas13; and (d) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the Cas13 guide RNA encoded by the first expression cassette.

In some embodiments, the first expression cassette comprises a constitutive promoter operably linked to a nucleic acid sequence encoding two or more Cas13 guide RNAs. For example, in some embodiments, the first expression cassette comprises a nucleic acid sequence encoding for 2, 3, 4, 5, 6, 7, 8, 9, or 10 distinct Cas13 guide RNAs. In some embodiments, the first expression cassette comprises a nucleic acid sequence encoding for at least 2, at least 3, at least 4, or at least 5 distinct Cas13 guide RNAs. In some embodiments, the first expression cassette comprises a nucleic acid sequence encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct Cas13 guide RNAs.

In some embodiments, a viral vector production system comprises a heterologous polynucleic acid encoding for Cas13a. In some embodiments, a viral vector production system comprises a heterologous polynucleic acid encoding for Cas13b. In some embodiments, a viral vector production system comprises a heterologous polynucleic acid encoding for Cas13c. In some embodiments, a viral vector production system comprises a heterologous polynucleic acid encoding for Cas13d.

In some embodiments, the Cas13 comprises the amino acid sequence of SEQ ID NO: 1 or an amino acid sequence of a functional variant having at least 80%, at least 85%, at least 90%, at least 95%, at least 96%, at least 97%, at least 98%, or at least 99% amino acid identity with the amino acid sequence of SEQ ID NO: 1.

In some embodiments, the first expression cassette further comprises the nucleic acid sequence encoding for Cas13. In some embodiments, the first expression cassette comprises a nucleic acid sequence of a constitutive promoter operably linked to both the nucleic acid sequence encoding the Cas13 gRNA and the nucleic acid sequence encoding for Cas13.

In some embodiments, the nucleic acid sequence of the payload molecule comprises: a 5′ UTR that comprises a target nucleic acid sequence that complements the Cas13 guide RNA encoded by the first expression cassette; a 3′ UTR that comprises a target nucleic acid sequence that complements the Cas13 guide RNA encoded by the first expression cassette; or a combination thereof.

In some embodiments, the nucleic acid sequence of the payload molecule comprises a tandem repeat of a target nucleic acid sequence that complements the Cas13 guide RNA encoded by the first expression cassette. In some embodiments, a tandem repeat may comprise at least 2, at least 3, at least 4, at least 5, at least 6, at least 7, at least 8, at least 9, or at least 10 copies of a target nucleic acid sequence. In some embodiments, a tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of a target nucleic acid sequence. In some embodiments, a tandem repeat comprises 2, 3, 4, 5, 6, 7, 8, 9, 10 copies of a target nucleic acid sequence.

In some embodiments, the first expression cassette comprises a tandem repeat of the nucleic acid sequence encoding the Cas13 guide RNA. In some embodiments, a tandem repeat may comprise at least 2, at least 3, at least 4, at least 5, at least 6, at least 7, at least 8, at least 9, or at least 10 copies of a nucleic acid sequence encoding the Cas13 guide RNA. In some embodiments, a tandem repeat comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 copies of a nucleic acid sequence encoding the Cas13 guide RNA. In some embodiments, a tandem repeat comprises 2, 3, 4, 5, 6, 7, 8, 9, 10 copies of a nucleic acid sequence encoding the Cas13 guide RNA.

In some embodiments, the first expression cassette comprises a nucleic acid sequence of two or more distinct Cas13 guide RNAs. For example, in some embodiments, the first expression cassette comprises a nucleic acid sequence encoding for 2, 3, 4, 5, 6, 7, 8, 9, or 10 distinct Cas13 guide RNAs. In some embodiments, the first expression cassette comprises a nucleic acid sequence encoding for at least 2, at least 3, at least 4, or at least 5 distinct Cas13 guide RNAs. In some embodiments, the first expression cassette comprises a nucleic acid sequence encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct Cas13 guide RNAs.

In some embodiments, the viral vector production component comprises one or more polynucleic acids that collectively encode the gene products of an AAV vector. In some embodiments, the viral vector component comprises the nucleic acid sequences of Rep52 or Rep40; Rep78 or Rep68; E2A; E4Orf6; VARNA; VP1; VP2; VP3; and AAP. In some embodiments, the viral terminal repeats of the transfer polynucleic acid are AAV inverted tandem repeats.

In some embodiments, the viral vector production component comprises one or more polynucleic acids that collectively encode the gene products of a lentivirus vector. In some embodiments, the viral vector component comprises the nucleic acid sequences of VSV-G, Gag-Pol, and Rev. In some embodiments, the viral terminal repeats of the transfer polynucleic acid are lentivirus long terminal repeats.

In some embodiments, the engineered cell comprises a viral vector production system as depicted in FIG. 4.

In some embodiments, at least one of the one or more of heterologous polynucleic acids is stably integrated into the genome of the engineered cell. In some embodiments, each of the one or more of heterologous polynucleic acids are stably integrated into the genome of the engineered cell.

In some embodiments, the engineered cell is derived from a HEK293 cell or a HeLa cell

### III. Methods of Reducing Expression of a Payload During Viral Vector Production

In some aspects, the disclosure relates to methods of reducing expression of a payload during viral vector production that utilizes an engineered cell described in Part II, wherein the payload comprises a target nucleic acid sequence (that complements the regulatory RNA encoded by the first expression cassette).

In some embodiments, wherein the engineered cell comprises an shRNA as a regulatory RNA, the method of reducing expression of a payload comprises expressing the shRNA during viral vector production.

In some embodiments, wherein the engineered cell comprises a nucleic acid sequence encoding Cas13 and a nucleic acid sequence encoding a Cas13 guide RNA, the method of reducing expression of a payload comprises expressing the Cas13 and the Cas13 guide RNA during viral vector production.

### EXAMPLES

### Example 1. shRNA-Mediated Knockdown of an AAV Payload Molecule

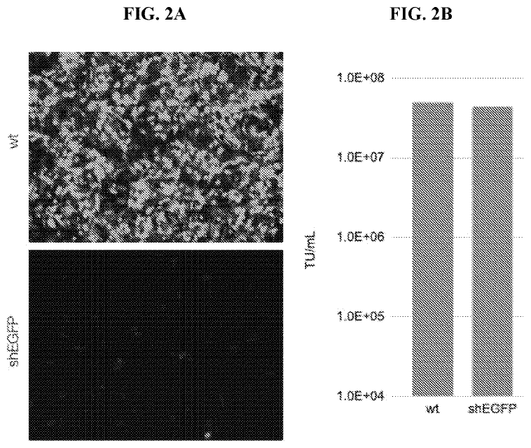

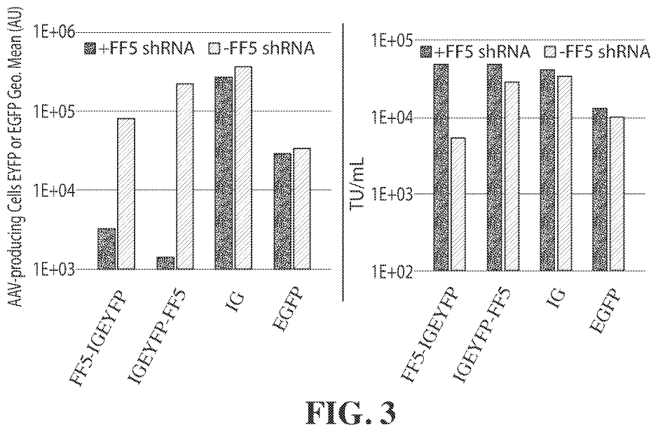

AAV pHelper, AAV pRepCap, and transfer plasmids were co-transfected with or without the shRNA expression plasmid against EGFP into HEK293FT cells. Three different shRNA plasmids were pooled together prior to testing (FIG. 1). 72 hours after transfection, AAV was harvested by three freeze thaw cycles in a dry ice isopropanol bath. Virus stock was serially diluted 1-, 10- and 100-fold and 10 uL of resulting viral stocks was transduced by addition to 5e4 HEK293FT cells plated in a 96-well plate. 48 hours after transduction, transduced cells were harvested and percentage of EGFP positive cells was determined by flow cytometry and used to calculate transducing units per mL (TU/mL). (FIGS. 2A-2B). Addition of EGFP shRNAs resulted in a significant decrease in EGFP fluorescence with only ˜1.1-fold reduction in AAV titers. AAV titers were not increased since EGFP has minimal toxicity and the minimal reduction in AAV titer shows that shRNAs had minimal effect on AAV production and packaging. Similar results were obtained when using FF5 shRNA 5 along with FF5 target sites on the transfer plasmid, along with a different Immunoglobulin (IG)-EYFP transfer sequence (FIG. 3).

TABLE 1
Exemplary shRNA sequences

| SEQ | | |
|---|---|---|
| ID NO | Description | Sequence |
| 2 | miRE.shRNA.T1 | CGACTTCTTAACCCAACAGAAGGCTCGAGAAGGTATATTGCTG |
| | | TTGACAGTGAGCGTAACGTACGCGGAATACTTCGAATAGTGA |
| | | AGCCACAGATGTATTCGAAGTATTCCGCGTACGTGTGCCTACT |
| | | GCCTCGGACTTCAAGGGGC |
| 3 | miRE.shRNA.T2 | CGACTTCTTAACCCAACAGAAGGCTCGAGAAGGTATATTGCTG |
| | | TTGACAGTGAGCGTTGCGTTGCTAGTACCAACCCTATAGTGAA |
| | | GCCACAGATGTATAGGGTTGGTACTAGCAACGCTTGCCTACTG |
| | | CCTCGGACTTCAAGGGGC |
| 4 | miRE.shRNA.FF3 | CGACTTCTTAACCCAACAGAAGGCTCGAGAAGGTATATTGCTG |
| | | TTGACAGTGAGCGTTACGATATGGGCTGAATACAAATAGTGA |
| | | AGCCACAGATGTATTTGTATTCAGCCCATATCGTTTGCCTACTG |
| | | CCTCGGACTTCAAGGGGC |
| 5 | miRE.shRNA.FF4 | CGACTTCTTAACCCAACAGAAGGCTCGAGAAGGTATATTGCTG |
| | | TTGACAGTGAGCGACGCTTGAAGTCTTTAATTAAATAGTGAAG |
| | | CCACAGATGTATTTAATTAAAGACTTCAAGCGGTGCCTACTGC |
| | | CTCGGACTTCAAGGGGC |
| 6 | miRE.shRNA.FF5 | CGACTTCTTAACCCAACAGAAGGCTCGAGAAGGTATATTGCTG |
| | | TTGACAGTGAGCGTAGCACTCTGATTTGACAATTATAGTGAAG |
| | | CCACAGATGTATAATTGTCAAATCAGAGTGCTTTGCCTACTGC |
| | | CTCGGACTTCAAGGGGC |
| 7 | miRE.shRNA.FF6 | CGACTTCTTAACCCAACAGAAGGCTCGAGAAGGTATATTGCTG |
| | | TTGACAGTGAGCGTTACCAAAGAGATTCCTCATAAATAGTGAA |
| | | GCCACAGATGTATTTATGAGGAATCTCTTTGGTTTGCCTACTGC |
| | | CTCGGACTTCAAGGGGC |
| 8 | miRE.shRNA.fluc. | CGACTTCTTAACCCAACAGAAGGCTCGAGAAGGTATATTGCTG |
| | 130 | TTGACAGTGAGCGTTACCAAAGAGATTCCTCATAAATAGTGAA |
| | | GCCACAGATGTATTTATGAGGAATCTCTTTGGTTTGCCTACTGC |
| | | CTCGGACTTCAAGGGGC |
| 9 | miRE.shRNA.fluc. | CGACTTCTTAACCCAACAGAAGGCTCGAGAAGGTATATTGCTG |
| | 563 | TTGACAGTGAGCGTTATGCACATATCGAGGTGGACATAGTGAA |
| | | GCCACAGATGTATGTCCACCTCGATATGTGCATCTGCCTACTG |
| | | CCTCGGACTTCAAGGGGC |
| 10 | miRE.shRNA.fluc. | CGACTTCTTAACCCAACAGAAGGCTCGAGAAGGTATATTGCTG |
| | 773 | TTGACAGTGAGCGTTGGACAAGACAATTGCACTGATTAGTGAA |
| | | GCCACAGATGTAATCAGTGCAATTGTCTTGTCCCTGCCTACTG |
| | | CCTCGGACTTCAAGGGGC |
| 11 | miRE.shRNA.fluc. | CGACTTCTTAACCCAACAGAAGGCTCGAGAAGGTATATTGCTG |
| | 1497 | TTGACAGTGAGCGTTTGGATTTCGAGTCGTCTTAATTAGTGAA |
| | | GCCACAGATGTAATTAAGACGACTCGAAATCCACTGCCTACTG |
| | | CCTCGGACTTCAAGGGGC |

TABLE 2
Targets of exemplary shRNA sequences

| SEQ | | |
|---|---|---|
| ID NO | Description | Sequence |
| 13 | T1 target | CACGTACGCGGAATACTTCGAA |
| 14 | T2 target | AGCGTTGCTAGTACCAACCCTA |
| 15 | FF3 target | AACGATATGGGCTGAATACAAA |
| 16 | FF4 target | CCGCTTGAAGTCTTTAATTAAA |
| 17 | FF5 target | AAGCACTCTGATTTGACAATTA |
| 18 | FF6 target | AACCAAAGAGATTCCTCATAAA |
| 19 | fluc.130 target | AACCAAAGAGATTCCTCATAAA |
| 20 | fluc.563 target | GATGCACATATCGAGGTGGACA |
| 21 | fluc.773 target | GGGACAAGACAATTGCACTGAT |
| 22 | fluc.1497 target | GTGGATTTCGAGTCGTCTTAAT |

### Example 2. Cas13-Mediated Knockdown of an AAV Payload Molecule

Cas13d can be employed to knockdown AAV payload GOI protein levels through RNA cleavage without modifying the GOI coding DNA sequence (FIG. 4). Cas13d does not exhibit a protospacer flanking sequence requirement allowing for targeting of any sequence within the transcribed region.

Exemplary Amino Acid Sequence for Cas13 (SEQ
ID NO: 1):
MSPKKKRKVEASIEKKKSFAKGMGVKSTLVSGSKVYMTTFAEGSDARLE
KIVEGDSIRSVNEGEAFSAEMADKNAGYKIGNAKFSHPKGYAVVANNPL
YTGPVQQDMLGLKETLEKRYFGESADGNDNICIQVIHNILDIEKILAEY
ITNAAYAVNNISGLDKDIIGFGKFSTVYTYDEFKDPEHHRAAFNNNDKL
INAIKAQYDEFDNFLDNPRLGYFGQAFFSKEGRNYIINYGNECYDILAL
LSGLRHWVVHNNEEESRISRTWLYNLDKNLDNEYISTLNYLYDRITNEL
TNSFSKNSAANVNYIAETLGINPAEFAEQYFRFSIMKEQKNLGFNITKL
REVMLDRKDMSEIRKNHKVFDSIRTKVYTMMDFVIYRYYIEEDAKVAAA
NKSLPDNEKSLSEKDIFVINLRGSFNDDQKDALYYDEANRIWRKLENIM
HNIKEFRGNKTREYKKKDAPRLPRILPAGRDVSAFSKLMYALTMFLDGK
EINDLLTTLINKFDNIQSFLKVMPLIGVNAKFVEEYAFFKDSAKIADEL
RLIKSFARMGEPIADARRAMYIDAIRILGTNLSYDELKALADTFSLDEN
GNKLKKGKHGMRNFIINNVISNKRFHYLIRYGDPAHLHEIAKNEAVVKF
VLGRIADIQKKQGQNGKNQIDRYYETCIGKDKGKSVSEKVDALTKIITG
MNYDQFDKKRSVIEDTGRENAEREKFKKIISLYLTVIYHILKNIVNINA
RYVIGFHCVERDAQLYKEKGYDINLKKLEEKGFSSVTKLCAGIDETAPD
KRKDVEKEMAERAKESIDSLESANPKLYANYIKYSDEKKAEEFTRQINR
EKAKTALNAYLRNTKWNVIIREDLLRIDNKTCTLFRNKAVHLEVARYVH
AYINDIAEVNSYFQLYHYIMQRIIMNERYEKSSGKVSEYFDAVNDEKKY
NDRLLKLLCVPFGYCIPRFKNLSIEALFDRNEAAKFDKEKKKVSGNSGS
GPKKKRKVAAAYPYDVPDYA

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A viral vector production system comprising:
(a) an engineered cell comprising a viral vector production component comprising one or more heterologous polynucleic acids that collectively encode the gene products of a viral vector;
(b) a heterologous nucleic acid sequence encoding for a first expression cassette, wherein the first expression cassette comprises a nucleic acid sequence of a constitutive promoter operably linked to a nucleic acid sequence encoding a regulatory RNA; and
(c) a transfer polynucleic acid comprising a central nucleic acid sequence flanked, on the 5′ and 3′ end, by a nucleic acid sequence of a viral terminal repeat.

2. The viral vector production system of claim 1, wherein the central nucleic acid sequence of the transfer polynucleic acid comprises a nucleic acid sequence encoding a multiple cloning site.

3. The viral vector production system of claim 2, wherein the central nucleic acid sequence comprises a multiple cloning sequence flanked by a target nucleic acid sequence that complements the regulatory RNA encoded by the first expression cassette.

4. The viral vector production system of claim 3, wherein the multiple cloning sequence is flanked by a tandem repeat of a target nucleic acid sequence that complements the regulatory RNA encoded by the first expression cassette.

5. The viral vector production system of claim 3 or claim 4, wherein the multiple cloning sequence is flanked on the 5′ end and the 3′ end by a target nucleic acid sequence that complements the regulatory RNA encoded by the first expression cassette.

6. The viral vector production system of any one of claims 2-5, wherein the central nucleic acid sequence further comprises a promoter.

7. The viral vector production system of any one of claims 1-6, wherein the central nucleic acid sequence of the transfer polynucleic acid sequence comprises a second expression cassette, wherein the second expression cassette comprises a nucleic acid sequence encoding a payload molecule operably linked to both a promoter and a target nucleic acid sequence that complements the regulatory RNA encoded by the first expression cassette.

8. The viral vector production system of claim 7, wherein the nucleic acid sequence of the payload molecule comprises: a 5′ UTR that comprises a target nucleic acid sequence that complements the regulatory RNA encoded by the first expression cassette; a 3′ UTR that comprises a target nucleic acid sequence that complements the regulatory RNA encoded by the first expression cassette; or a combination thereof.

9. The viral vector production system of claim 7 or claim 8, wherein the nucleic acid sequence of the payload molecule comprises a tandem repeat of a target nucleic acid sequence that complements the regulatory RNA encoded by the first expression cassette.

10. The viral vector production system of any one of claims 1-9, wherein the first expression cassette comprises a tandem repeat of the nucleic acid sequence encoding the regulatory RNA.

11. The viral vector production system of any one of claims 1-10, wherein the first expression cassette comprises a nucleic acid sequence of two or more distinct regulatory RNAs.

12. The viral vector production system of any one of claims 1-11, wherein the first expression cassette further comprises a nucleic acid sequence encoding a gene product of the viral vector production component.

13. The viral vector production system of claim 12, wherein the nucleic acid sequence encoding the gene product in the first expression cassette has: a 5′ UTR comprising the nucleic acid sequence encoding the regulatory RNA; an intron comprising the nucleic acid sequence encoding the regulatory RNA; a 3′ UTR comprising the nucleic acid sequence encoding the regulatory RNA; or a combination thereof.

14. The viral vector production system of any one of claims 1-13, wherein the first expression cassette further comprises a nucleic acid sequence encoding a selectable marker.

15. The viral vector production system of claim 14, wherein the selectable marker comprises a fluorescent protein or antibiotic resistance protein.

16. The viral vector production system of claim 14 or claim 15, wherein the nucleic acid sequence encoding the selectable maker in the first expression cassette has a 5′ UTR comprising the nucleic acid sequence encoding the regulatory RNA; an intron comprising the nucleic acid sequence encoding the regulatory RNA; a 3′ UTR comprising the nucleic acid sequence encoding the regulatory RNA; or a combination thereof.

17. The viral vector production system of any one of claims 1-16, wherein the viral vector production system is an AAV viral vector production system, wherein the viral vector production component comprises one or more polynucleic acids that collectively encode the gene products of an AAV vector.

18. The viral vector production system of claim 17, wherein the viral vector component comprises the nucleic acid sequences of Rep52 or Rep40; Rep78 or Rep68; E2A; E4Orf6; VARNA; VP1; VP2; VP3; and AAP.

19. The viral vector production system of claim 17 or claim 18, wherein the viral terminal repeats of the transfer polynucleic acid are AAV inverted tandem repeats.

20. The viral vector production system of any one of claims 1-16, wherein the viral vector production system is a lentivirus vector production system, wherein the viral vector production component comprises one or more polynucleic acids that collectively encode the gene products of a lentivirus vector.

21. The viral vector production system of claim 20, wherein the viral vector component comprises the nucleic acid sequences of VSV-G, Gag-Pol, and Rev.

22. The viral vector production system of claim 20 or claim 21, wherein the viral terminal repeats of the transfer polynucleic acid are lentivirus long terminal repeats.

23. The viral vector production system of any one of claims 1-22, wherein at least one of the one or more of heterologous polynucleic acids of the viral vector production component is stably integrated into the genome of the engineered cell.

24. The viral vector production system of any one of claims 1-23, wherein each of the one or more of heterologous polynucleic acids of the viral vector production component is stably integrated into the genome of the engineered cell.

25. The viral vector production system of any one of claims 1-24, wherein the engineered cell further comprises the heterologous nucleic acid sequence encoding for the first expression cassette.

26. The viral vector production system of any one of claims 1-25, wherein the engineered cell is derived from a HEK293 cell, a HeLa cell, a BHK cell or a Sf9 cell.

27. The viral vector production system of any one of claims 1-26, wherein the regulatory RNA is an shRNA or an amiRNA.

28. The viral vector production system of claim 27, wherein the nucleic acid sequence encoding the shRNA comprises a nucleic acid sequence of any one of SEQ ID NOs: 2-11.

29. The viral vector production system of claim 27 or claim 28, wherein the first expression cassette comprises a nucleic acid sequence encoding for a selectable marker, wherein the nucleic acid sequence encoding for the selectable marker comprises an intron having, from 5′ to 3′: (i) an intron donor site; (ii) a nucleic acid sequence encoding for the shRNA or amiRNA; and (iii) an intron acceptor site.

30. The viral vector production system of claim 29, wherein the intron comprises a tandem repeat, an shRNA cluster, or an amiRNA cluster of the nucleic acid sequence encoding for the shRNA or amiRNA.

31. The viral vector production system of claim 29 or claim 30, wherein the nucleic acid sequence encoding for the selectable marker comprises: a 5′ UTR, wherein the intron of the selectable marker is located in the 5′ UTR; a 3′UTR, and wherein the intron of the selectable marker is located in the 3′ UTR; or a combination thereof.

32. The viral vector production system of any one of claims 29-31, wherein the intron of the selectable marker is located in the coding region of the nucleic acid sequence encoding for the selectable marker.

33. The viral vector production system of any one of claims 28-31, wherein the intron comprises the nucleic acid sequence of SEQ ID NO: 12.

34. The viral vector production system of any one of claims 1-26, wherein the regulatory RNA is a Cas13 guide RNA.

35. The viral vector production system of claim 34, wherein the first expression cassette comprises a constitutive promoter operably linked to a nucleic acid sequence encoding two or more Cas13 guide RNAs.

36. The viral vector production system of claim 34 or claim 35, further comprising a heterologous polynucleic acid encoding for Cas13.

37. The viral vector production system of claim 36, wherein the Cas13 is Cas13d.

38. The viral vector production system of claim 37, wherein Cas13d comprises the amino acid sequence of SEQ ID NO: 1 or an amino acid sequence having at least 80% identity to the amino acid sequence of SEQ ID NO: 1.

39. The viral vector production system of any one of claims 36-38, wherein heterologous polynucleic acid encoding for Cas13 further comprises the first expression cassette.

40. The viral vector production system of claim 39, wherein the first expression cassette comprises a nucleic acid sequence of a constitutive promoter operably linked to both the nucleic acid sequence encoding the Cas13 gRNA and the nucleic acid sequence encoding for Cas13.

41. The viral vector production system of any one of claims 36-40, wherein the engineered cell further comprises the heterologous polynucleic acid encoding for Cas13.

42. An engineered cell for viral vector production comprising one or more heterologous polynucleic acids collectively comprising:
(a) a viral vector production component collectively encoding the gene products of a viral vector;
(b) a first expression cassette, wherein the first expression cassette comprises a nucleic acid sequence of a constitutive promoter operably linked to a nucleic acid sequence encoding an shRNA or amiRNA; and
(c) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the shRNA or amiRNA encoded by the first expression cassette.

43. The engineered cell of claim 42, wherein the nucleic acid sequence of the payload molecule comprises: a 5′ UTR that comprises a target nucleic acid sequence that complements the shRNA or amiRNA encoded by the first expression cassette; a 3′ UTR that comprises a target nucleic acid sequence that complements the shRNA or amiRNA encoded by the first expression cassette; or a combination thereof.

44. The engineered cell of claim 42 or claim 43, wherein the nucleic acid sequence of the payload molecule comprises a tandem repeat of a target nucleic acid sequence that complements the shRNA or amiRNA encoded by the first expression cassette.

45. The engineered cell of any one of claims 42-44, wherein the first expression cassette comprises a tandem repeat, shRNA cluster or amiRNA cluster of the nucleic acid sequence encoding the shRNA or amiRNA.

46. The engineered cell of any one of claims 42-45, wherein the first expression cassette comprises a nucleic acid sequence of two or more distinct shRNAs or two or more distinct amiRNAs.

47. The engineered viral vector production system of any one of claims 42-45, wherein the first expression cassette comprises a nucleic acid sequence encoding for a selectable marker, wherein the nucleic acid sequence encoding for the selectable marker comprises an intron having, from 5′ to 3′: (i) an intron donor site; (ii) a nucleic acid sequence encoding for the shRNA or amiRNA; and (iii) an intron acceptor site.

48. The engineered cell of claim 47, wherein the intron comprises a tandem repeat shRNA cluster or amiRNA cluster of the nucleic acid sequence encoding for the shRNA or amiRNA.

49. The engineered cell of claim 47 or claim 48, wherein nucleic acid sequence encoding for the selectable marker comprises: a 5′ UTR, wherein the intron of the selectable marker is located in the 5′ UTR; a 3′UTR, wherein the intron of the selectable marker is located in the 3′ UTR; or a combination thereof.

50. The engineered cell of any one of claims 47-49, wherein the intron of the selectable marker is located in the coding region of the nucleic acid sequence encoding for the selectable marker.

51. The engineered cell of any one of claims 47-50, wherein the intron comprises the nucleic acid sequence of SEQ ID NO: 12.

52. The engineered cell of any one of claims 42-50, wherein the nucleic acid sequence encoding the shRNA comprises the nucleic acid sequence of any of SEQ ID NOs: 2-11.

53. The engineered cell of any one of claims 42-52, wherein the viral vector production component comprises one or more polynucleic acids that collectively encode the gene products of an AAV vector.

54. The engineered cell of claim 53, wherein the viral vector component comprises the nucleic acid sequences of Rep52 or Rep40; Rep78 or Rep68; E2A; E4Orf6; VARNA; VP1; VP2; VP3; and AAP.

55. The engineered cell of 53 or claim 54, wherein the viral terminal repeats of the transfer polynucleic acid are AAV inverted tandem repeats.

56. The engineered cell of any one of claims 42-52, wherein the viral vector production component comprises one or more polynucleic acids that collectively encode the gene products of a lentivirus vector.

57. The engineered cell of claim 56, wherein the viral vector component comprises the nucleic acid sequences of VSV-G, Gag-Pol, and Rev.

58. The engineered cell of claim 56 or claim 57, wherein the viral terminal repeats of the transfer polynucleic acid are lentivirus long terminal repeats.

59. The engineered cell of any one of claims 42-58, wherein at least one of the one or more of heterologous polynucleic acids is stably integrated into the genome of the engineered cell.

60. The engineered cell of any one of claims 42-59, wherein each of the one or more of heterologous polynucleic acids are stably integrated into the genome of the engineered cell.

61. The engineered cell of any one of claims 42-60, wherein the engineered cell is derived from a HEK293 cell a HeLa cell, a BHK cell or a Sf9 Cell.

62. A method of reducing expression of a payload molecule during viral vector production in the engineered cell any one of claims 42-60, comprising expressing the shRNA or amiRNA during viral vector production.

63. An engineered cell for viral vector production comprising one or more heterologous polynucleic acids collectively comprising:
(a) a viral vector production component collectively encoding the gene products of a viral vector;
(b) a first expression cassette, wherein the first expression cassette comprises a nucleic acid sequence of a constitutive promoter operably linked to a nucleic acid sequence encoding a Cas13 guide RNA;
(c) a nucleic acid sequence encoding Cas13; and
(d) a transfer polynucleic acid comprising a central nucleic acid sequence flanked, on the 5′ and 3′ end, by a nucleic acid sequence of a viral terminal repeat, wherein the central nucleic acid sequence comprises a nucleic acid sequence encoding a payload molecule operably linked to both a promoter and a target nucleic acid sequence that complements the Cas13 guide RNA encoded by the first expression cassette.

64. The engineered cell of claim 63, wherein the first expression cassette comprises a constitutive promoter operably linked to a nucleic acid sequence encoding two or more Cas13 guide RNAs.

65. The engineered cell of claim 63 or claim 64, wherein the Cas13 is Cas13d.

66. The engineered cell of claim 65, wherein Cas13d comprises the amino acid sequence of SEQ ID NO: 1 or an amino acid sequence having at least 80% identity with the amino acid sequence of SEQ ID NO: 1.

67. The engineered cell of any one of claims 63-65, wherein the first expression cassette further comprises the nucleic acid sequence encoding for Cas13.

68. The engineered cell of claim 67, wherein the first expression cassette comprises a nucleic acid sequence of a constitutive promoter operably linked to both the nucleic acid sequence encoding the Cas13 gRNA and the nucleic acid sequence encoding for Cas13.

69. The engineered cell of any one of claims 63-68, wherein the nucleic acid sequence of the payload molecule comprises: a 5′ UTR that comprises a target nucleic acid sequence that complements the Cas13 guide RNA encoded by the first expression cassette; a 3′ UTR that comprises a target nucleic acid sequence that complements the Cas13 guide RNA encoded by the first expression cassette; or a combination thereof.

70. The engineered cell of any one of claims 63-69, wherein the nucleic acid sequence of the payload molecule comprises a tandem repeat of a target nucleic acid sequence that complements the Cas13 guide RNA encoded by the first expression cassette.

71. The engineered cell of any one of claims 63-70, wherein the first expression cassette comprises a tandem repeat of the nucleic acid sequence encoding the Cas13 guide RNA.

72. The engineered cell of any one of claims 63-71, wherein the first expression cassette comprises a nucleic acid sequence of two or more distinct Cas13 guide RNAs.

73. The engineered cell of any one of claims 63-72, wherein the viral vector production component comprises one or more polynucleic acids that collectively encode the gene products of an AAV vector.

74. The engineered cell of claim 73, wherein the viral vector component comprises the nucleic acid sequences of Rep52 or Rep40; Rep78 or Rep68; E2A; E4Orf6; VARNA; VP1; VP2; VP3; and AAP.

75. The engineered cell of 73 or claim 74, wherein the viral terminal repeats of the transfer polynucleic acid are AAV inverted tandem repeats.

76. The engineered cell of any one of claims 63-72, wherein the viral vector production component comprises one or more polynucleic acids that collectively encode the gene products of a lentivirus vector.

77. The engineered cell of claim 76, wherein the viral vector component comprises the nucleic acid sequences of VSV-G, Gag-Pol, and Rev.

78. The engineered cell of claim 76 or claim 77, wherein the viral terminal repeats of the transfer polynucleic acid are lentivirus long terminal repeats.

79. The engineered cell of any one of claims 63-78, wherein at least one of the one or more of heterologous polynucleic acids is stably integrated into the genome of the engineered cell.

80. The engineered cell of any one of claims 63-79, wherein each of the one or more of heterologous polynucleic acids are stably integrated into the genome of the engineered cell.

81. The engineered cell of any one of claims 63-80, wherein the engineered cell is derived from a HEK293 cell, a HeLa cell, a BHK cell or a Sf9 Cell.

82. A method of reducing expression of a payload molecule during viral vector production in the engineered cell any one of claims 63-81, comprising expressing the Cas13 and the Cas13 guide RNA during viral vector production.


SEQUENCE LISTING (23 sequences)

SEQ ID NO: 1 - protein, 1000 aa, Artificial Sequence
  Synthetic
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 2 - DNA, 147 bp, Artificial Sequence
  Synthetic
cgacttcttaacccaacagaaggctcgagaaggtatattgctgttgacagtgagcgtaacgtacgcggaatacttcgaatagtgaagccacagatgtattcgaagtattccgcgtacgtgtgcctactgcctcggacttcaaggggc

SEQ ID NO: 3 - DNA, 147 bp, Artificial Sequence
  Synthetic
cgacttcttaacccaacagaaggctcgagaaggtatattgctgttgacagtgagcgttgcgttgctagtaccaaccctatagtgaagccacagatgtatagggttggtactagcaacgcttgcctactgcctcggacttcaaggggc

SEQ ID NO: 4 - DNA, 147 bp, Artificial Sequence
  Synthetic
cgacttcttaacccaacagaaggctcgagaaggtatattgctgttgacagtgagcgttacgatatgggctgaatacaaatagtgaagccacagatgtatttgtattcagcccatatcgtttgcctactgcctcggacttcaaggggc

SEQ ID NO: 5 - DNA, 146 bp, Artificial Sequence
  Synthetic
cgacttcttaacccaacagaaggctcgagaaggtatattgctgttgacagtgagcgacgcttgaagtctttaattaaatagtgaagccacagatgtatttaattaaagacttcaagcggtgcctactgcctcggacttcaaggggc

SEQ ID NO: 6 - DNA, 146 bp, Artificial Sequence
  Synthetic
cgacttcttaacccaacagaaggctcgagaaggtatattgctgttgacagtgagcgtagcactctgatttgacaattatagtgaagccacagatgtataattgtcaaatcagagtgctttgcctactgcctcggacttcaaggggc

SEQ ID NO: 7 - DNA, 147 bp, Artificial Sequence
  Synthetic
cgacttcttaacccaacagaaggctcgagaaggtatattgctgttgacagtgagcgttaccaaagagattcctcataaatagtgaagccacagatgtatttatgaggaatctctttggtttgcctactgcctcggacttcaaggggc

SEQ ID NO: 8 - DNA, 147 bp, Artificial Sequence; identical to SEQ ID NO: 7
  Synthetic

SEQ ID NO: 9 - DNA, 147 bp, Artificial Sequence
  Synthetic
cgacttcttaacccaacagaaggctcgagaaggtatattgctgttgacagtgagcgttatgcacatatcgaggtggacatagtgaagccacagatgtatgtccacctcgatatgtgcatctgcctactgcctcggacttcaaggggc

SEQ ID NO: 10 - DNA, 147 bp, Artificial Sequence
  Synthetic
cgacttcttaacccaacagaaggctcgagaaggtatattgctgttgacagtgagcgttggacaagacaattgcactgattagtgaagccacagatgtaatcagtgcaattgtcttgtccctgcctactgcctcggacttcaaggggc

SEQ ID NO: 11 - DNA, 147 bp, Artificial Sequence
  Synthetic
cgacttcttaacccaacagaaggctcgagaaggtatattgctgttgacagtgagcgtttggatttcgagtcgtcttaattagtgaagccacagatgtaattaagacgactcgaaatccactgcctactgcctcggacttcaaggggc

SEQ ID NO: 12 - DNA, 41 bp, Artificial Sequence
  Synthetic
  misc_feature (9)..(12): n is a, c, g, or t
aggtaagtnnnntactttaggacccttttttttccacaggt

SEQ ID NO: 13 - DNA, 22 bp, Artificial Sequence
  Synthetic
cacgtacgcggaatacttcgaa

SEQ ID NO: 14 - DNA, 22 bp, Artificial Sequence
  Synthetic
agcgttgctagtaccaacccta

SEQ ID NO: 15 - DNA, 22 bp, Artificial Sequence
  Synthetic
aacgatatgggctgaatacaaa

SEQ ID NO: 16 - DNA, 22 bp, Artificial Sequence
  Synthetic
ccgcttgaagtctttaattaaa

SEQ ID NO: 17 - DNA, 22 bp, Artificial Sequence
  Synthetic
aagcactctgatttgacaatta

SEQ ID NO: 18 - DNA, 22 bp, Artificial Sequence
  Synthetic
aaccaaagagattcctcataaa

SEQ ID NO: 19 - DNA, 22 bp, Artificial Sequence
  Synthetic
aaccaaagagattcctcataaa

SEQ ID NO: 20 - DNA, 22 bp, Artificial Sequence
  Synthetic
gatgcacatatcgaggtggaca

SEQ ID NO: 21 - DNA, 22 bp, Artificial Sequence
  Synthetic
gggacaagacaattgcactgat

SEQ ID NO: 22 - DNA, 22 bp, Artificial Sequence
  Synthetic
gtggatttcgagtcgtcttaat

SEQ ID NO: 23 - DNA, 888 bp, Unknown
  AAV2
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa